(19) United States
(12) Patent Application Publication
(10) Pub. No.: US 2026/0188426 A1
(43) Pub. Date: Jul. 2, 2026

(54) RESOLUTION OF VARIANTS OF UNCERTAIN SIGNIFICANCE

(71) Applicant: Helix, Inc., San Mateo, CA (US)

(72) Inventors: Kelly Schiabor Barrett, San Diego, CA (US); Alexandre Bolze, San Francisco, CA (US); James Lu, San Mateo, CA (US); Elizabeth Cirulli Rogers, Lakeside, CA (US); Sophie Candille, San Mateo, CA (US); Matthew Levy, Washington, DC (US); Ildiko Thibodeau, San Mateo, CA (US)

(21) Appl. No.: 19/002,038

(22) Filed: Dec. 26, 2024

(51) Int. Cl. G16B 20/20; G16H 10/60; G16H 15/00; G16H 20/10; G16H 50/30; G16H 50/70

(52) U.S. Cl. CPC G16B 20/20; G16H 10/60; G16H 15/00; G16H 20/10; G16H 50/30; G16H 50/70

(57) ABSTRACT

Systems and methods for enhancing classification of variants. One embodiment is a method comprising retrieving genetic information for a patient that calls a variant within a gene related to a medical condition, retrieving classification data for the variant, identifying a cohort of patients that have the variant, retrieving health record data related to the medical condition for the patients in the cohort, and identifying a prevalence of the medical condition within the cohort. When the classification data conflicts with a prevalence of the medical condition within the cohort, the method includes generating a recommendation to apply a classification to the variant that conforms with the prevalence of the medical condition within the cohort. When the classification data does not conflict, the method includes generating a recommendation to confirm a classification for the variant. The method includes providing a recommendation to treat the patient based on the classification for the variant.

### FIELD

The disclosure relates to the field of genomics, and in particular, to interpretation of genetic variants.

### BACKGROUND

Variant interpretation within the field of genomics remains a difficult and complex process. Determining the impact of a specific variant upon a specific person may be hard to determine, particularly for rare variants. This often results in different laboratories providing different interpretations for the same variant. It also leads to many variants without a clear interpretation, also known as Variants of Uncertain Significance (VUS). When clinical tests or results return a VUS classification for a variant, this fails to provide actionable insights to clinical care providers. Thus, resolving VUS is an important aspect of improving healthcare.

Variant scientists presently use guidelines provided by the American College of Medical Genetics (ACMG) and/or Association of Molecular Pathology (AMP) to select and weigh the value of various publications, studies, and articles when classifying the impact of a variant. In many circumstances, the variants under consideration are rare, which means that variant scientists have access to an extremely limited set of data when classifying the variant. For example, some variants may have previously been encountered only a few times during diagnostic testing, or may not have been previously observed at all, resulting in increased uncertainty and subjectivity, even when ACMG and/or AMP guidelines are carefully followed.

Clinical laboratories and health care providers therefore continue to seek out new, robust solutions that are data-driven and consistent when classifying genetic variants.

### SUMMARY

Embodiments described herein leverage large population databases, also known as “all-comers cohorts,” that include comprehensive genetic and phenotypic data for a group of patients assembled without selection bias. This is notably different from current clinical labs that either leverage publications or their own database of patients that have been tested for a genetic condition, as those two sources are biased towards patients with a diagnosis related to the genetic condition being considered. Specifically, the present invention uses existing Electronic Health Record (EHR) data for a general patient population that has undergone sequencing as part of a widespread sequencing campaign. By combining EHR and sequencing data from this population to dynamically create reference cases, an unbiased data set is created that may be used to confirm or revise variant classifications. The statistical prevalence of linked health conditions within these reference cases is then used to drive and/or revise variant classification.

One embodiment is a system for enhancing classification of variants in the field of genetics. The system includes an interface able to retrieve genetic information that corresponds with a patient and that calls a variant within a gene related to a medical condition, and to retrieve classification data for the variant, and a controller able to identify a cohort of patients that have the variant and are not included within the classification data, retrieve health record data related to the medical condition for the patients in the cohort, and identify a prevalence of the medical condition within the cohort. The controller is further able in an event that the classification data conflicts with a prevalence of the medical condition within the cohort, to generate a recommendation to apply a classification to the variant that conforms with the prevalence of the medical condition within the cohort. The controller is further able, in an event that the classification data does not conflict with a prevalence of the medical condition within the cohort, to generate a recommendation to confirm a classification for the variant recited within the classification data. The controller is still further able to provide a recommendation to treat the patient based on the classification for the variant.

A further embodiment is a method for enhancing classification of variants in the field of genetics. The method includes retrieving genetic information that corresponds with a patient and that calls a variant within a gene related to a medical condition, retrieving classification data for the variant, identifying a cohort of patients that have the variant, retrieving health record data related to the medical condition for the patients in the cohort, and identifying a prevalence of the medical condition within the cohort. In an event that the classification data conflicts with a prevalence of the medical condition within the cohort, the method further includes generating a recommendation to apply a classification to the variant that conforms with the prevalence of the medical condition within the cohort. In an event that the classification data does not conflict with a prevalence of the medical condition within the cohort, the method further includes generating a recommendation to confirm a classification for the variant recited within the classification data. The method further includes providing a recommendation to treat the patient based on the classification for the variant.

A further embodiment is a non-transitory computer readable medium embodying programmed instructions which, when executed by a processor, are operable for performing a method for enhancing classification of variants in the field of genetics. The method includes retrieving genetic information that corresponds with a patient and that calls a variant within a gene related to a medical condition, retrieving classification data for the variant, identifying a cohort of patients that have the variant, retrieving health record data related to the medical condition for the patients in the cohort, and identifying a prevalence of the medical condition within the cohort. In an event that the classification data conflicts with a prevalence of the medical condition within the cohort, the method further includes generating a recommendation to apply a classification to the variant that conforms with the prevalence of the medical condition within the cohort. In an event that the classification data does not conflict with a prevalence of the medical condition within the cohort, the method further includes generating a recommendation to confirm a classification for the variant recited within the classification data. The method further includes providing a recommendation to treat the patient based on the classification for the variant.

Other illustrative embodiments (e.g., methods and computer-readable media relating to the foregoing embodiments) may be described below. The features, functions, and advantages that have been discussed can be achieved independently in various embodiments or may be combined in yet other embodiments, further details of which can be seen with reference to the following description and drawings.

### DESCRIPTION OF THE DRAWINGS

Some embodiments of the present disclosure are now described, by way of example only, and with reference to the accompanying drawings. The same reference number represents the same element or the same type of element on all drawings.

FIG. 1 is a diagram depicting a sample processing architecture in an illustrative embodiment.

FIG. 2 is a block diagram illustrating an architecture to process genomics information in an illustrative embodiment.

FIG. 3 is a flowchart depicting a method for enhancing classification of variants (e.g., by resolving VUS classifications), based on population data from an all-comers cohort in an illustrative embodiment.

FIG. 4 is a flowchart depicting a method for utilizing multiple control sets of patients to enhance classification of variants in an illustrative embodiment.

FIG. 5 is a flowchart depicting a method for utilizing phenotype scoring to enhance classification of variants in an illustrative embodiment.

FIG. 6 is a block diagram depicting categories of health record data in an illustrative embodiment.

FIG. 7 is a block diagram depicting variant classification data in an illustrative embodiment.

FIG. 8 is a table that summarizes sequencing data for patients and is maintained at a genomics server in an illustrative embodiment.

FIG. 9 is a table that summarizes variant data for patients and is maintained at a genomics server in an illustrative embodiment.

FIG. 10 is a table that summarizes biomarker test data for patients and is maintained at a genomics server in an illustrative embodiment.

FIGS. 11-12 depict Graphical User Interfaces (GUIs) that facilitate the communication of information related to variant classifications in illustrative embodiments.

FIG. 13 depicts an illustrative computing system operable to execute programmed instructions embodied on a computer readable medium.

### DESCRIPTION

The figures and the following description depict specific illustrative embodiments of the disclosure. It will thus be appreciated that those skilled in the art will be able to devise various arrangements that, although not explicitly described or shown herein, embody the principles of the disclosure and are included within the scope of the disclosure. Furthermore, any examples described herein are intended to aid in understanding the principles of the disclosure, and are to be construed as being without limitation to such specifically recited examples and conditions. As a result, the disclosure is not limited to the specific embodiments or examples described below, but by the claims and their equivalents.

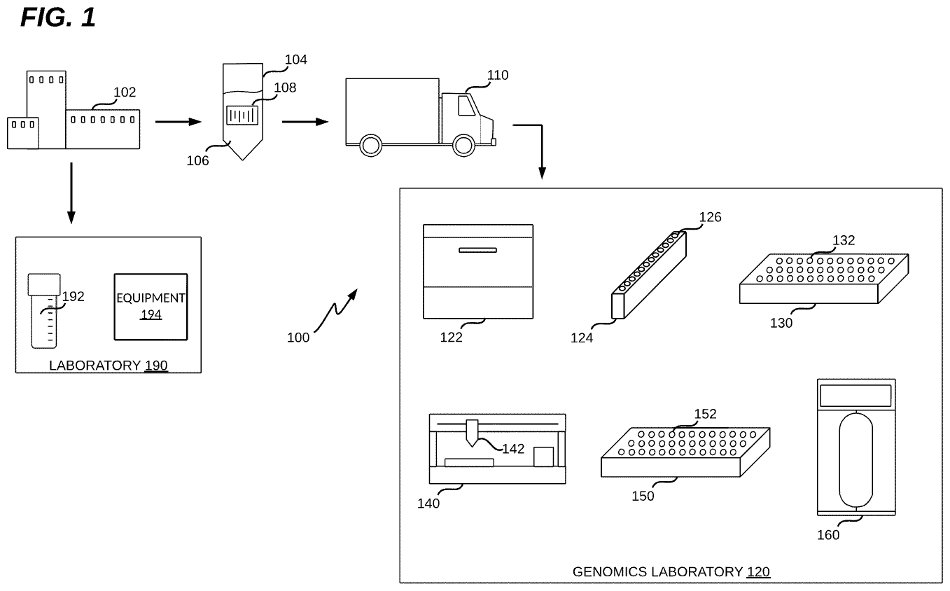

FIG. 1 is a diagram depicting a sample processing architecture 100 in an illustrative embodiment. Sample processing architecture 100 comprises any system or organizational structure for acquiring and sequencing biological samples in a high-volume, high-throughput manner. Sample processing architecture 100 may be utilized, for example, to collect and sequence genetic material (in the form of Deoxyribonucleic Acid (DNA) or Ribonucleic Acid (RNA)) found within thousands or tens of thousands of samples 106 daily, via multiple healthcare provider networks 102.

Healthcare provider networks 102 may comprise hospitals, clinics, practitioner offices, laboratories, surgical centers, etc., that engage in or facilitate the practice of medicine. In one embodiment, healthcare provider networks 102 each comprise groups of hospitals that treat millions of patients. As a part of the practice of medicine, healthcare provider networks 102 acquire samples 106 for sequencing. For example, a healthcare provider network 102 may acquire samples 106 as part of a population screening program, as part of medical treatment, etc. In further embodiments, patients within a healthcare provider network 102 receive sampling kits for independent, self-directed use in acquiring samples 106. The specific amount of sequencing desired for a sample 106 may comprise a selected set of one or more genes, an exome, the entire genome of a patient, etc. The samples 106 are stored in sample containers 104, which may be accompanied by Customer Sample Identifiers (CSIs) 108. A delivery service 110 provides the samples 106 to a genomics laboratory 120 for processing.

Healthcare provider networks 102 may also acquire samples 192 for conventional blood testing (described below). These samples 192 may be provided to laboratory 190 for analysis via equipment 194 (e.g., a chemically treated test strip, biochemical assay, etc.), or may be analyzed via at-home testing methods. For example, a patient may utilize an at-home device to measure blood sugar levels, which are then collected as health record data for the patient. Sample processing architecture 100 provides a technical benefit by allowing laboratory 190 and genomics laboratory 120 to specialize in different methods of analysis.

Procedures within genomics laboratory 120 related to genetics may include accessioning, sample plating, storage, extraction, library preparation, enrichment, and sequencing processes. These processes acquire genetic material from a sample 106, separate the genetic material from other constituents, duplicate the genetic material, and quantify the genetic material order to determine a swathe of sequence data, such as an exome or entire genome for a subject (e.g., a human patient, an organelle of a human patient, etc.). Although the procedures discussed herein are specific with regard to one method of sequencing, other techniques may be utilized in accordance with known standards in order to perform sequencing for samples 106. For example, although certain short-read technologies herein are discussed as utilizing hybridization capture techniques, amplicon-based techniques may be used alternatively or to supplement those techniques. Long-read techniques may also or alternatively be utilized.

### Accessioning

Accessioning refers to receiving and preparing samples 106 for later laboratory processes. In one embodiment, accessioning includes receiving a batch of samples 106 (e.g., hundreds or thousands of samples 106) from one or more delivery services 110 each day for processing. For example, packages that each include tens or hundreds of samples 106 may be delivered to genomics laboratory 120 via the United States Postal Service (USPS), or a private package carrier.

Each sample 106 may be retained within a sample container 104, such as a five milliliter (mL) test tube. In this embodiment, the sample container 104 is sealed to prevent the sample 106 from being exposed to the environment and also to prevent the sample 106 from co-mingling with other samples 106. For example, the sample 106 may be sealed via a cap that is threaded, glued, press-fit, etc. At the time of delivery, the sample container 104 may further include a remnant of a sampling tool, such as a portion of a swab that was utilized to acquire the sample.

In many embodiments, a CSI 108 for the sample 106 is reported via a component affixed to or integrated with the sample container 104. The CSI 108 uniquely distinguishes the sample 106 from other samples 106 being received. For example, a CSI 108 may uniquely distinguish a sample 106 from other samples 106 in the same batch, other samples 106 received on the same date, other samples 106 received from the same healthcare provider network 102, etc. A CSI 108 may be reported via a barcode label, Quick Response (QR) code label, Radio Frequency Identifier (RFID) chip, or any suitable visual, transmission-generating, or other physical component or marking affixed to or integrated with the sample container 104.

In further embodiments, the sample container 104 is itself sealed within an external container such as a bag (not shown). Using an external container helps to prevent contamination, by ensuring that a technician at the genomics laboratory 120 does not contact biological material from the sample 106 that may exist on an outer surface of the sample container 104. Use of an external container may also be required by law (e.g., Department of Transportation (DOT) guidelines). Use of an external container additionally helps to prevent cross-contamination between samples 106. Furthermore, in embodiments where samples 106 may include blood or a pathogen, an external container provides an additional barrier to protect the health of technicians. The external container may additionally include documentation confirming the CSI 108, information for the subject that the sample was sourced from, and/or information indicating circumstances of sampling. The circumstances of sampling may include, for example, a sampling date, a sampling method, a location that the sample was acquired, a name or title for a person who performed the sampling, and/or additional notes.

In this embodiment, the sample 106 comprises a chemical solution. For example, the sample 106 may comprise a prepared aqueous solution such as a saline solution, or may comprise a bodily fluid such as blood, saliva, mucus, etc. In some embodiments, each of the samples 106 fills between two and five milliliters of volume within its corresponding sample container 104. In further embodiments, the samples 106 may be constituted at the genomics laboratory 120 from dried blood spots applied to filter paper, may comprise buccal material, etc.

The samples 106 further include genetic material such as Deoxyribonucleic Acid (DNA), Ribonucleic Acid (RNA), etc. In many instances, the genetic material is one of many constituent components within the sample 106. For example, the genetic material may exist within the nuclei of white blood cells that are included within the sample 106. In a further example, genetic material may exist within viruses or bacteria within the sample 106. In this embodiment, the genetic material is not yet isolated from the remaining constituent components of the sample 106.

After receipt of the samples 106, batches of the samples 106 (e.g., as stored within sample containers 104 and/or external containers) may be heated in ovens 122 to facilitate cell lysis. The temperature and duration of heating, may be chosen such that pathogenic material within the samples 106 is rendered harmless, or such that cellular lysis occurs. For example, heating may occur at a temperature in a range of about forty and eighty (e.g., fifty) degrees Celsius (C), for a period of time in a range of about fifteen and two hundred (e.g., thirty) minutes. In some embodiments, including embodiments where the samples 106 are primarily the contents of a blood draw, the heating step may be foregone.

In this embodiment, upon completion of heating, the batches of samples 106 are removed from the ovens 122. In one embodiment, sample containers 104 are removed from corresponding external containers, such as by cutting the external containers open. With the sample containers 104 now available for direct interaction, the sample containers 104 are inspected. As a part of this process, a technician or automated system may determine a CSI 108 for a sample 106, and may compare the CSI 108 to a CSI 108 listed on documentation provided in the external container. If there is a discrepancy between the CSI 108 on the sample container 104 and the CSI 108 listed in the documentation, the sample 106 may be flagged as having an error condition. Similarly, if the CSI 108 on the sample container 104 is damaged (e.g., abraded, heat-damaged, or water-damaged) or has become unreadable, the sample 106 may be flagged as having an error condition.

A technician or automated system may further inspect the contents of the sample container 104, via visual or other methods. If the sample 106 does not include an expected constituent component (or is otherwise non-compliant), then the sample 106 is flagged as having an error condition. For example, if the sample 106 is primarily saliva and includes a fluid that is not permitted (e.g., blood), includes an entire swab or no swab, appears to have a fractured or broken casing, or is outside of an expected range of volume (e.g., between two and five milliliters), then the sample 106 may be flagged as having an error condition.

Samples 106 that have not been flagged as having an error condition proceed to sample integration. In one embodiment, as a part of sample integration, the sample 106 is assigned a Laboratory Sample Identifier (LSI). The LSI uniquely identifies the sample 106 from other samples 106 received for the batch, received on the same day, processed in the same laboratory, and/or handled by the same organization performing sequencing. In many embodiments, the LSI is stored in a memory of a genomics server (e.g., within a laboratory sample database), and is uniquely associated with a corresponding CSI 108 for the sample. The LSI may also be associated with any error conditions reported for the sample 106.

In many embodiments, CSIs 108 originally provided with the samples 106 are in the form of a paper barcode. In such embodiments, the paper barcode may be printed in aqueous ink. This renders the barcode subject to degradation upon exposure to liquid in the laboratory environment, which is undesirable.

To ensure that each sample container 104 is capable of traveling through the genomics laboratory 120 without its identifier being physically degraded, a corresponding LSI may be indicated at the sample container 104. The LSI may be indicated via the application of a barcode label, Quick Response (QR) code label, Radio Frequency Identifier (RFID) chip, or other visual, transmission-generating, or other physical component affixed to or integrated with the sample container.

In one embodiment, the LSI is printed onto a barcode label comprising rip-proof material (e.g., vinyl) in a water-insoluble ink. This implementation ensures that the barcode label is resistant to physical and chemical degradation. The barcode may be applied around an entire perimeter of the sample container 104, ensuring that the sample container 104 may be scanned from any angle.

In further embodiments, the element used to report the LSI is accompanied by a visually-distinct mark that enables rapid confirmation by a technician that the sample 106 has been integrated into the laboratory environment. The visually-distinct mark may comprise a colored ring (e.g., around an entire perimeter of the sample container), a logo, a physical feature, a stamp, etc.

### Sample Plating

With the samples 106 having been successfully integrated into the environment of the genomics laboratory 120, the samples 106 are ready for analytics to be performed. To this end, the samples 106 are prepared for transfer to a sample microplate 130. The sample microplate 130 may be labeled with a unique identifier via similar techniques to those used for sample containers 104 above. The unique identifier distinguishes the sample microplate 130 from other sample microplates 130. In one embodiment, the sample microplate 130 comprises a solid body defining three hundred and eighty-four wells, distributed across sixteen rows and twenty-four columns, each well having a capacity of between thirty and one hundred microliters. In a further embodiment, the sample microplate 130 comprises a solid body defining ninety-six wells, distributed across eight rows and twelve columns, each well having a capacity of between one hundred and three hundred microliters. Any suitable number and arrangement of wells may be selected as a matter of design choice.

As a part of preparing the samples 106 for transfer to the sample microplate 130, a technician may place sample containers 104 onto a rack 124, and scan each sample container 104 to determine an LSI for each location 126 (e.g., each container receptacle) on the rack 124. In some embodiments, the rack 124 is assigned a unique identifier that distinguishes it from other racks 124. The rack 124 may be labeled with a unique identifier using techniques similar to those used for sample containers 104. The technician, or automated machinery such as a server operating an optical scanner, may then associate the unique identifier for the rack 124, along with the locations 126 assigned to the samples 106, with the corresponding LSIs of the samples 106 stored at the rack 124.

The technician additionally unseals the sample containers 104. Unsealing of sample containers 104 may be a deeply labor-intensive process, particularly when laboratory processes are performed at scale to handle tens of thousands of samples 106 per day. Thus, a technician may utilize automated tooling to enhance the speed at which sample containers 104 are unsealed. The tooling may, for example, lever open, pull, unscrew, cut, or drill each sample container 104, in order to make the sample 106 within available for physical transfer to the sample microplate 130.

One or more racks 124 of samples 106 are provided to a Liquid Handler (LH) 140, such as an automated robot that operates an end effector 142 in accordance with one or more Numerical Control (NC) programs to transfer liquids between wells via arrays of micropipettes. An LH 140 is also known as a “Liquid Handling System”. LH 140 may comprise, for example, a Hamilton Microlab Star Liquid Handling System.

In this embodiment, the LH 140 proceeds to transfer a portion of each sample 106 at a rack 124 to a well 132 within the sample microplate 130. The well 132 is not shared with (i.e., is distinct from) wells 132 for other samples 106. For example, the well 132 for each sample 106 may be predetermined in accordance with a control program used by the genomics laboratory 120. In one embodiment, the LH 140 transfers the portions of the samples 106 to the wells 132 of the sample microplate 130 by providing instructions to actuators, piezoelectric elements, and/or pressure systems operating the end effector 142. In such an embodiment, the end effector 142 may align its array of micropipettes with the sample containers 104 to retrieve portions of the samples 106. Furthermore, in such an embodiment, the end effector 142 may dynamically align its array of micropipettes with the sample microplate 130 to deposit the portions of the samples 106 at the wells 132.

Because there is a known relationship between locations 126 at the rack 124 and wells 132 of the sample microplate 130 (e.g., as indicated by row and column), contents of the memory of a genomics server (e.g., a laboratory sample database) may be updated to indicate the well 132 storing genetic material for each sample 106. In one embodiment, the memory is further updated to associate a unique identifier for the sample microplate 130 with the samples 106 stored therein.

In one embodiment, programmed instructions for the LH 140 may direct the end effector 142 to position itself above a set of disposable tips, descend into the tips to attach the tips, reposition the end effector 142 above the rack of sample containers 104, adjust spacing between micropipettes within the array, descend until the tips reach the sample containers 104, draw liquid from the sample containers 104, deposit the liquid into a well 132 at the sample microplate 130, and then dispose of the tips. Such a process may be repeated across sample containers 104 stored on multiple racks until the sample microplate 130 is filled with portions from the samples 106. In one embodiment, one or more wells 132 on the sample microplate 130 are filled with a control reagent instead of a portion of a sample 106.

The amount of liquid drawn from each sample container 104 may comprise a small fraction of the overall volume of the sample container 104. For example, an amount of liquid drawn may comprise several microliters, such as between two and ten microliters. Upon completion of transfer from the sample containers 104 to the wells, the sample microplate 130 may be covered with a liquid and/or gas-impermeable layer, such as foil or paraffin. Sample containers 104 remaining on the racks may be resealed, for example with pressure-fit caps having a color distinct from an original color for the sample containers. With accessioning now complete for the sample microplate 130, the sample microplate 130 is transferred to a next section of the laboratory for processing.

In embodiments where the genomics laboratory 120 performs both short-read and long-read sequencing workflows, the sample plating techniques discussed above may be performed separately, asynchronously, and/or in parallel for short-read technologies (e.g., via an Illumina sequencing platform such as a NovaSeq X) and for long-read technologies (e.g., via a PacBio sequencing platform such as a Revio). These techniques may also vary between long-read sequencing workflows and short-read sequencing workflows. For example, the number and nature of plates used for samples 106, the amount of sample 106 used for the sequencing workflow, and whether a process is manual or automated all may vary between sequencing workflows. For example, these differences may occur in the workflows to support the requirements of different pieces of sequencing equipment 160, to account for differences in sequencing volume between workflows, etc. Samples 106 received at the genomics laboratory 120 may include sufficient genetic material to support multiple sequencing processes (e.g., both short-read and long-read sequencing processes). Thus, in many embodiments, samples 106 provide genetic material for both short-read and long-read sequencing, supporting the rigor of diagnostic genetic testing processes.

### Storage

In one embodiment, accessioned samples 106, samples 106 ready for analytics, and/or samples 106 that have already been sequenced, are stored for later use. For example, samples 106, sample containers 104, and/or sample microplates 130 may be stored at room temperature, or may be cryogenically frozen at a low temperature (e.g., negative eighty degrees Celsius) within a freezer and arranged in racks for later retrieval. Samples 106 may be preserved for periods of days or years, enabling rapid re-testing to be performed for subjects without the need for re-acquiring genetic material. Storage of the samples 106 provides notable value in the event that contents of a well 132 used for sequencing do not meet with rigorous quality control standards. Specifically, storage enables re-sampling to occur in the event that there is a desire to re-sequence a sample 106.

### Extraction

Sample microplates 130 are transferred to a portion of the genomics laboratory 120 dedicated to extraction of the genetic material. The segment of the genomics laboratory 120 that performs extraction and other pre-amplification operations may be sealed from, and/or positively pressurized relative to, other portions of the genomics laboratory 120.

During extraction, a sample microplate 130 is acquired and provided to an LH 140. The LH 140 that performs extraction may be different from the LH 140 that performs sample plating. The LH 140 may apply a reagent to each well 132 that lyses cells within each well. For example, this may be performed in order to lyse white blood cells containing genetic material for a human, or may comprise lysing other types of cells or components to expose other types of genetic material. The reagents used for pre-amplification processes may be stored at the LH 140 in a temperature-controlled manner, and may even be vibrated or mixed on a regular basis to ensure that the reagents are evenly distributed in suspension.

In one embodiment, extraction further includes an LH 140 aspirating and dispensing reagents that selectively bind to genetic material released from the lysed cells. This process may include applying a bead (not shown) to the well 132. In one embodiment, the beads comprise magnetic beads that selectively bind to the genetic material (e.g., DNA). This allows for isolation and purification of the genetic material while contaminants remain in solution. In one embodiment, the magnetic bead is drawn to a magnetic base at or under the sample microplate 130. After the genetic material has been drawn to the bead, and after the bead has been secured to the base of the well, a flushing step may be performed where remaining fluid in each well is washed away. This ensures that potential impurities are removed from the well 132. The LH 140 may further add or remove fluid from each well 132 to perform additional concentration and/or elution of the genetic material, and may transfer fluid from the wells 132 of the sample microplate 130 to wells 152 of a genome stock microplate 150. The genome stock microplate 150 may be labeled with a unique identifier, and the contents of each well 152 of the genome stock microplate 150 may be associated with a corresponding LSI. In all phases of operation, the LH 140 is operated to ensure that fluid is not transferred between wells 152, as this results in contamination by intermingling genetic material for different samples 106.

In one embodiment, a portion of fluid is removed from each well 152 of the genome stock microplate 150 for quality control purposes. Concentration of genetic material within the wells 152 may be confirmed via testing of this fluid, such as by application of a dye that reacts with the genetic material at known levels of fluorescence for known concentrations.

In embodiments where the genomics laboratory 120 performs both short-read and long-read sequencing workflows, the extraction techniques discussed above may be performed separately, asynchronously, and/or in parallel for short-read technologies (e.g., via an Illumina sequencing platform such as a NovaSeq X) and for long-read technologies (e.g., via a PacBio sequencing platform such as a Revio).

### Library Preparation

After extraction is completed, library preparation may be performed for the contents of the genome stock microplate 150. The bead for each well 152, including ionically bonded genetic material, is transferred to a distinct well of a library preparation microplate (not shown). The library preparation microplate includes an identifier that uniquely distinguishes it from other library preparation microplates, and the LSI associated with each well 152 on the genome stock microplate 150 may be mapped to a corresponding well on the library preparation microplate.

The library preparation microplate may be transferred to a new portion of the genomics laboratory 120 that is sealed from, and/or positively pressurized relative to, other portions of the genomics laboratory 120 that do not perform amplification of genetic material. This feature helps to prevent amplified genetic material from entering portions of the laboratory where genetic material has not been amplified, which could result in contamination. The transfer process may be performed by placing a library preparation microplate into an airlock at the pre-amplification portion of the genomics laboratory 120, sealing the airlock, and then retrieving the library preparation microplate from the airlock via the amplification portion of the genomics laboratory 120.

In one embodiment, a reagent is applied to each well of the library preparation microplate. The reagent ionically bonds to the surface of the bead within the well, and does so more strongly than the genetic material. This releases the genetic material from the surface of the bead of each well, enabling the genetic material to be chemically interacted with.

Library preparation may include normalization of a concentration of genetic material in each well of the library preparation microplate. Library preparation further includes fragmentation of the genetic material via an enzyme or via the application of physical forces. During this process, the entire genome (e.g., roughly three billion base pairs for a human genome), may be fragmented into pieces. In one embodiment where short-read sequencing is performed, the pieces vary between three hundred and four hundred base pairs in length. These pieces are known as nucleic acid fragments. In a further embodiment where long-read sequencing is performed, the pieces may vary between five hundred and fifty thousand or more base pairs in length.

In one embodiment utilizing short-read sequencing, the nucleic acid fragments undergo adaptor ligation and indexing in accordance with known techniques. For example, this may comprise Next Generation Sequencing (NGS) library preparation processes defined by Illumina. Next, a limited amount of Polymerase Chain Reaction (PCR) amplification is performed upon the library. The resulting solution is then purified and eluted via operation of an LH 140.

During library preparation, one or more reference samples of genetic material, distinct from the genetic material found in the samples, may be added to wells of the library preparation microplate. The reference samples do not include genetic material received from a customer, but rather include known sequences of base pairs. The reference samples serve as controls to ensure that processes are carried out with sufficient quality.

Upon completion of library preparation, desired fragments of the genetic material (e.g., thousands or millions of distinct fragments of the genetic material, each corresponding with a different portion of a genome of the subject) have been ligated to predefined adapters (e.g., DNA adapters) that bind with the genetic material. Each of the adaptor-ligated fragments is referred to as a “library”.

In further embodiments, the probes applied to each well of the library preparation plate include chemical identifiers (colloquially referred to as “barcodes”) that are distinct from each other. The use of a different chemical identifier for probes applied to each well of the library preparation microplate enables sequencing to later be performed for multiple subjects on the same flow cell, without conflating sequencing results for those subjects.

In one embodiment utilizing long-read sequencing, library preparation may be performed via physical shearing of DNA to achieve a target size distribution mode between ten and twenty-five kilobases (kb), such as between fifteen and eighteen kb. The resulting nucleic acid fragments may be coupled to adapters to prepare them for sequencing via Single-Molecule Sequencing in Real Time (SMRT) or other long-read technologies.

The library preparation processes discussed herein may further comprise controlling a concentration of the genetic material in each well, and purification and/or elution of the resulting material. Similar to the processes performed after extraction of genetic material, concentration of genetic material after library preparation may be confirmed for each well via testing.

### Enrichment

After library preparation, enrichment processes may be performed in order to either directly amplify (e.g., via amplicon or multiplexed PCR) or capture (e.g., via hybrid capture) predefined libraries. This enhances the ease of sequencing desired portions of the genome. In some embodiments, enrichment is foregone for long-read sequencing processes.

In one embodiment, during enrichment, customized biotinylated oligonucleotide probes are applied to the libraries. The probes selectively hybridize genetic material occupying desired portions of the genome for the genetic material, such as specific genes, or the entire exome. Magnetic beads bind to biotin molecules in the probes to attach the hybridized material to the magnetic beads. Magnetic forces capture the beads in place, enabling remaining fluid within each well to be removed or washed out, thereby removing impurities and leaving only the genetic material that is desired. Genetic material may be released from the beads in a similar manner to that discussed above for prior processes.

In a further embodiment, hybrid capture target enrichment is performed. During this process, the probes comprise tailored oligonucleotides that are chosen to bind to the genetic material. The range of probes may be tailored as a group to bind to specific alleles, specific genes, the exome, the entire genome, etc. That is, each probe may bind to a nucleic acid fragment at a specific location on the genome, and the range of probes may be selected to ensure that alleles, genes, the exome, or the entire genome of the subject being considered is acquired. Utilizing probes in this manner may enhance efficiency of the sequencing process, by foregoing sequencing of all of the roughly three billion base pairs found in the human genome.

The enrichment process may further comprise controlling a concentration of the genetic material in each well, and purification and/or elution of the resulting material. Similar to the processes performed after extraction of genetic material, concentration of genetic material after enrichment may be confirmed for each well via testing.

### Sequencing

Sequencing may be performed according to any of a variety of techniques, including short-read and long-read techniques, via sequencing equipment 160 (e.g., an Illumina NovaSeq X sequencing machine, a PacBio Revio sequencing machine, etc.). As used herein, short-read sequencing refers to sequencing technologies that generate reads of five hundred or fewer base pairs in length. Short-read sequencing may be used as the basis for “synthetic long read” technologies that stitch individual short reads together, but as used herein, short-read sequencing does not refer to the creation or use of synthetic long reads.

In one embodiment, short-read sequencing is performed as Sequencing by Synthesis (SBS). For example, sets of enriched libraries of genetic material bound to probes in earlier steps may be transferred to a flow cell, and annealed to oligonucleotide probes within the flow cell. At this stage, the contents of multiple wells may be applied to the same flow cell, because the libraries within those wells are tagged with the chemical identifiers referred to above. In one embodiment, the chemical identifiers comprise nucleotide sequences that are detectable during the sequencing process to determine a corresponding LSI.

Complementary sequences may then be created via enzymatic extension to create a double-stranded portion of genetic material. The double-stranded genetic material may then be denatured, and the library fragment may be washed away. Bridge amplification may then be performed to create copies of the remaining molecule in a localized cluster. For example, a cluster may comprise twenty to fifty copies of the same molecule, localized to a location the size smaller than a pinhead on the flow cell.

In this embodiment, sequencing primers are annealed to library adapters in order to prepare the flow cell for SBS. During SBS, the sequencing primer uses reverse terminator fluorescent oligonucleotides, one base per cycle, for a number of cycles (e.g., one hundred and fifty cycles) in the forward direction. After the addition of each nucleotide, clusters are excited by a light source, resulting in fluorescence that can be measured. The emission wavelength and signal intensity for each cluster determines a base call for that cluster. Fluorescent moieties are then flushed from the flow cell. A chemical group blocking a 3′ end of the fragment is then removed, enabling a subsequent nucleotide to be read. This tightly controls nucleotide addition and detection.

Additionally in this embodiment, base calls across cycles at the same physical location on the flow cell occur at the same cluster, and hence indicate sequential reads for copies of the same fragment of the genetic material. After each cycle, denaturing and annealing are performed to extend the index primer. A complementary reverse strand is created and extended via bridge amplification. The reverse strand is then read in the reverse direction for a number of cycles, in a manner similar to reads in the forward direction.

Depending on whether a complete human genome or another set of genomic data is being tested, different reagents (e.g., probes, primers, etc.) may be chosen. That is, different reagents and/or processes may be utilized for library preparation for a pathogen (e.g., bacteria, virus) or an organelle (e.g., mitochondria) than for a human genome. Pathogens exhibiting Ribonucleic Acid (RNA) genomes may have their genetic material translated to DNA before sequencing, enrichment, and/or library preparation are performed, via known techniques, such as Next Generation Sequencing (NGS) techniques.

In a further embodiment, long-read sequencing (e.g., sequencing of nucleic acid fragments larger than one kilobase) is performed in an SMRT process, where nucleic acid fragments are circularized and bound to a DNA polymerase enzyme. The bound pair enter a sequencing chamber, and the DNA polymerase adds complementary bases to the DNA strand that are fluorescently labeled to result in different colors for different bases.

As labelled bases are added by the polymerase, the color of the base is recorded, and then the fluorescent label is removed. The next base for the circularized nucleic acid fragment is then added and recorded, iteratively, until the circularized nucleic acid fragment has been sequenced a desired number of times.

Throughout the processes discussed above, the laboratory environment may be carefully controlled to ensure quality. For example, temperature within each segment of the laboratory may be carefully monitored and controlled, and ultraviolet lighting or other features capable of inactivating genetic material may be carefully positioned to ensure that contamination does not occur.

### Bioinformatics

Sequencing data may be stored in any suitable format. In one embodiment, raw sequencing data generated during short-read sequencing is stored in a file format, such as Binary Base Call (BCL). This raw data may be fed to an analytical pipeline, such as a cloud-based computing environment. Raw sequencing data may be processed by the pipeline into a second format, such as a text-based FASTQ format, that reports quality scores. The second format may then be analyzed to perform alignment of sequence reads to a reference genome, such as a reference genome reported in a Browser Extensible Data (BED) file. The aligned sequence data may be reported as a Binary Alignment Map (BAM) file or Compressed Reference-oriented Alignment Map (CRAM) file. In one embodiment, long-read sequencing data is output from the corresponding sequencing machine as one or more BAM files, obviating the need for long-read sequence data undergoing the conversion processes discussed above.

The aligned sequence data may then be called, resulting in a Variant Call Format (VCF) file reporting called variants at each location of the genome that was sequenced, together with secondary metrics, such as quality indicator metrics. As used herein, a variant comprises a unique combination of genetic information, in the form of consecutive base pairs at a specific set of locations (e.g., genomic coordinates) along a portion of a chromosome or other genomic segment. Each variant is distinguished from other variants by having a different combination of base pairs along the set of locations. This may be due to Single Nucleotide Polymorphisms (SNPs) which relate to common single nucleotide changes, Single Nucleotide Variants (SNVs) which relate to rare nucleotide changes, insertions and/or deletions (Indels) which relate for example to the insertion or deletion of less than thirty base pairs, or differing numbers of repetitions, Copy Number Variants (CNVs), which relate to larger insertions or deletions, translocations, inversions, other types of genetic variants, or even combinations of variants, such as haplotypes or Multi-nucleotide variants (MNVs).

The called sequence data may be provided to a data analyst via a User Interface (UI), such as a Graphical User Interface (GUI) presented via a display. The technician may then validate the resulting called sequence data and release it for reporting to subjects, health care providers, and/or scientists.

### Architecture to Process Genomics Information

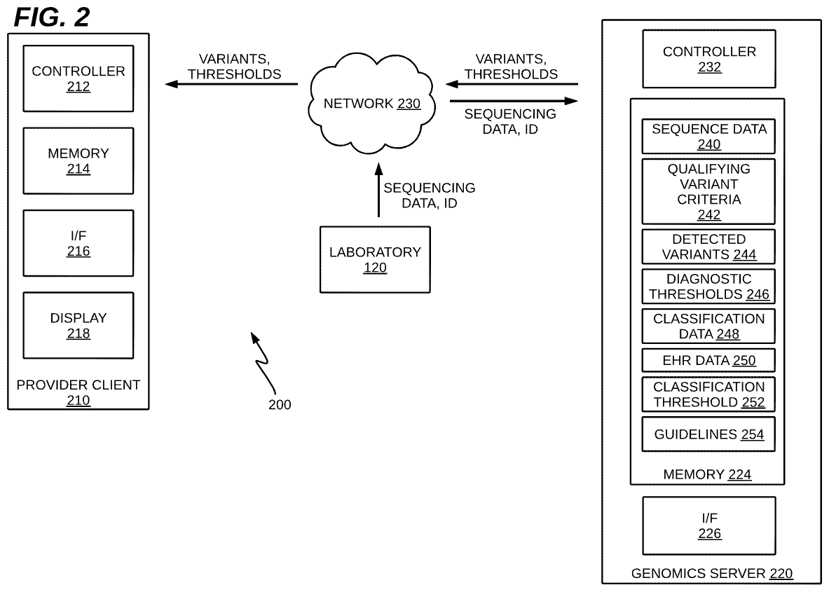

FIG. 2 is a block diagram illustrating a genomics architecture 200 in an illustrative embodiment. Genomics architecture 200 comprises any combination of systems and devices operable to review, process, and/or control access to sequencing data, including sequencing data received from genomics laboratory 120. In this embodiment, genomics architecture 200 comprises a genomics server 220 which receives sequencing data and identifiers (e.g., CSIs 108, LSIs, etc.) from genomics laboratory 120, via network 230. The sequencing data received and processed by the genomics server 220 may be supplied for multiple different types of sequencing operations, including short-read and long-read sequencing operations.

Genomics server 220 receives the sequencing data via interface (I/F) 226, such as an Ethernet interface, wireless interface compliant with Institute of Electrical and Electronics Engineers (IEEE) 802.11 standards, or other physical interface capable of transmitting and receiving digital data. The sequence data 240 is stored in memory 224 for the population of patients (e.g., millions of patients) that have been sequenced by genomics laboratory 120, and may be maintained in any suitable format. Examples of such formats include CRAM, VCF, BAM, and others. Memory 224 may store, for example, sequence data 240 describing multiple patients, and this sequence data 240 may be maintained in a de-identified format to facilitate the advancement of research. Memory 224 may be implemented via a cloud storage service, or may comprise a storage medium, such as a hard disk or flash memory device.

Memory 224 may additionally store qualifying variant criteria 242, detected variants 244, and diagnostic thresholds 246 for diagnosis and/or treatment of specific diseases. For example, a diagnostic threshold 246 may recite one or multiple criteria for diagnosis, which may include genetic as well as other factors. In one embodiment, the portion of memory 224 storing these components is distinct from the portion of memory 224 storing sequence data 240. In one embodiment, memory 224 additionally stores classification data 248, comprising classifications of variants (e.g., according to ACMG/AMP guidelines, or based on historically applied classifications for variants). Classification data 248 includes variant classifications that have previously been assigned to specific genetic variants, such as by a clinical laboratory.

In a further embodiment, memory 224 additionally stores Electronic Health Record (EHR) data 250 for one or more patients. The EHR data 250 may comprise EHR data that has been rendered into a uniform format, such as an Observational Medical Outcomes Partnership OMOP format, and may comprise health records for each patient that sequencing data has been stored for. Memory 224 further includes classification threshold 252, which indicates a degree of certainty (e.g., as reflected by a p-value or odds ratio) that is required before establishing that a variant is significantly more prevalent in affected individuals compared to control groups. As used herein, both “individuals” and “patients” include persons who are within a healthcare provider network 102, and include healthy persons. This information can later be applied to attribute the ACMG PS4 criteria for a variant, and be used for revising or enhancing the confidence of variant classification (e.g., according to ACMG/AMP guidelines). For example, in circumstances where a variant classified as a VUS has been enriched by new cases and controls within EHR data 250, the classification threshold 252 may be used to determine that sufficient PS4 evidence has been met for classifying the variant as likely pathogenic. Guidelines 254 in memory 224 reflect standards of care, medical practices, and/or contextual information for medical conditions related to variants being classified.

Controller 232 manages the operations of genomics server 220, and may for example analyze sequence data 240 to determine alignments to a reference genome, identify detected variants 244, control access and authentication related to sequence data 240, communicate with one or more provider clients 210, and/or perform additional operations. Controller 232 may be implemented, for example, as custom circuitry, as a hardware processor executing programmed instructions, as a combination of shared hardware processing resources implementing a compute service, or some combination thereof.

Genomics architecture 200 further comprises provider client 210, which is configured to receive information regarding detected variants 244 and/or diagnostic thresholds 246. In this embodiment, provider client 210 includes a controller 212, a memory 214, an interface (I/F) 216, and a display 218. Controller 212 manages the operations of the provider client 210, and may be implemented, for example, as custom circuitry, as a hardware processor executing programmed instructions, or some combination thereof. Memory 214 comprises information for interpreting the data received via I/F 216. Display 218 may comprise a screen, projector, etc., for presenting information to a user of provider client 210.

In further embodiments, one or more genomics servers are utilized. For example, a first genomics server may facilitate the storage and analysis of sequencing data from genomics laboratory 120, while a second genomics server may retrieve sequence data to facilitate interactions with a provider client 210 in order to resolve variants of uncertain significance.

### Resolving Variants of Uncertain Significance

After sequencing data for a patient has been acquired, it is capable of being utilized for automation-assisted interpretation of variants, such as by controller 232 of genomics server 220. As used herein, automation-assisted interpretation of variants refers to utilizing large population databases, that combine clinical and genomic records, in order to revise or enhance the confidence of variant classification (e.g., according to ACMG and/or AMP guidelines).

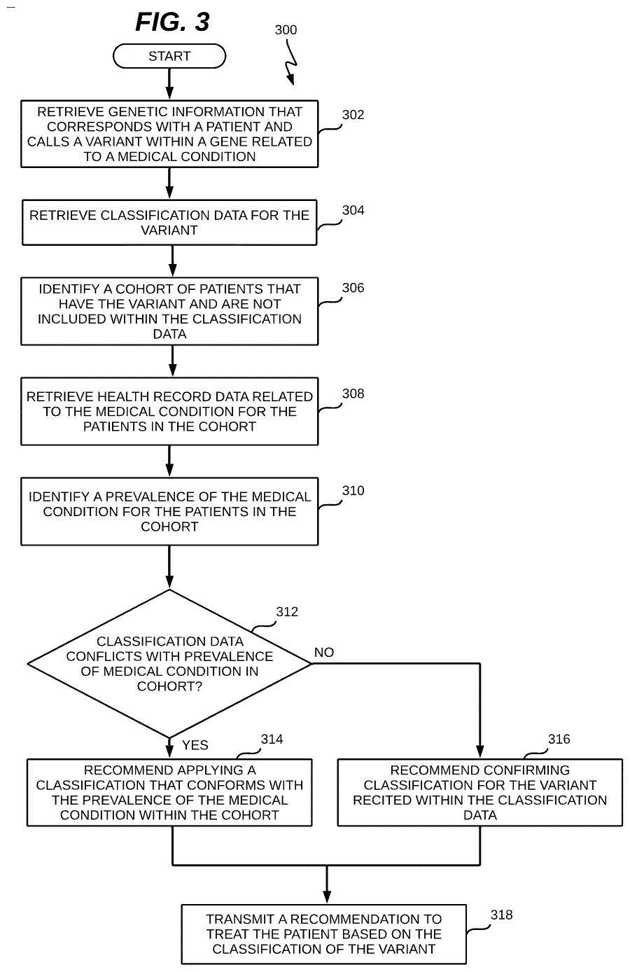

FIG. 3 is a flowchart depicting a method 300 for enhancing classification of variants (e.g., by resolving VUS classifications), based on population data from an all-comers cohort. The steps of the flow charts described herein are not all inclusive and may include other steps not shown, and the steps may be performed in an alternative order. For example, method 300 may be performed serially or in parallel on a massive scale for each of many patients within a genomics server 220 hosting sequence data 240 for hundreds of thousands or millions of patients.

Step 302 comprises interface (I/F) 226 of genomics server 220 retrieving genetic information (e.g., sequence data 240) that corresponds with a patient and that calls a variant within a gene related to a medical condition. As used herein, a “medical condition” refers to a disease or phenotype for a patient. The medical conditions considered via this process are capable of being reported either explicitly or implicitly within medical records for patients. For example, the medical condition may be associated with one or more codes defined by a medical vocabulary, or one or more measurements. In one embodiment, the genetic information is reported in a Variant Call Format (VCF) file for the patient, which is maintained within sequence data 240 and/or detected variants 244 at memory 224.

Step 304 comprises I/F 226 retrieving classification data 248 for the variant. Classification data 248 comprises a list of classifications that have historically been applied to the variant (e.g., by a clinical laboratory). Illustrative classifications for a variant include pathogenic (PATH), likely pathogenic (LPATH), VUS, likely benign (LBEN), and benign (BEN). A single variant may have historically received multiple classifications, often because different clinical laboratories had different pieces of evidence available to make these interpretations. In this case, the classification is often labeled as ‘CONFLICTING INTERPRETRATIONS’ using data sources such as ClinVar.

The classification data 248 may be sourced, for example, from a clinical laboratory, as a published set of historical classifications or other source that reports results from a population of patients who are expected to either have the medical condition or a potentially pathogenic variant related to the condition. That is, the classifications in classification data 248 are expected to either exhibit potential selection bias, or to be small enough in number such that enhancement by comparison to the general population is desired.

In one embodiment, the classification data 248 for the variant is selected for enhancement via steps 306-316 if the classification data 248 reports the variant as a VUS, or includes multiple classifications for the variant (e.g., VUS for one group of patients, LPATH for another group of patients, etc.), a situation which is also referred to as “conflicting interpretations”. In further embodiments, the classification data 248 for the variant is chosen for enhancement regardless of a classification of the variant. In still further embodiments, the classification data 248 is chosen for enhancement if the variant has been classified few times (i.e., less than a threshold number of times, such as for a small number of patients).

Step 306 comprises controller 232 of genomics server 220 identifying a cohort of patients that have the variant. The patients within the cohort are not included within the classification data 248 that was previously retrieved. That is, the cohort of patients are not the same patients as those already reported within the classification data 248. Instead, the cohort of patients include new patients having the variant, who have not been previously studied/reviewed in relation to the variant. In one embodiment, patients that have the variant are identified via batched review of specific portions of VCF files for those patients. This step is notable because instead of considering patients who have been historically classified for a specific variant, it draws on population data to find a group of patients that have the variant, regardless of whether it was previously classified.

As used herein, patients that “have the variant” are patients having variant calls that reflect similar changes to a reference genome (e.g., GRCh38) as the variant being classified. Depending on the embodiment, this may include patients having the exact same variation in nucleotides from the reference genome as the patient, patients having a copy number or structural variant that is the same as the variant being classified, a coding change that results in the same change to protein structure as for the variant being classified, a variation that results in a predicted loss of function of the same protein as for the variant being classified, etc.

The patients in the cohort are selected from an “all comers population”. Because patients for the cohort are chosen from a balanced and diverse population (i.e., not just patients expected to have genetic risk relating to the gene), the cohort of patients helps to address potential selection bias found within the classification data 248. For example, patients may be selected from one or more general patient populations across one or more healthcare provider networks 102.

Step 306 is therefore innovative in that it flips the process of variant interpretation from a focus on prior-studied patients, to the general patient population. This in turn changes the entire schema for performing variant classification in a manner that is both statistically rigorous and insightful.

Step 308 comprises controller 232 retrieving health record data related to the medical condition for the patients in the cohort. In one embodiment, the health record data comprises Electronic Health Record (EHR) data. The health record data may include medical vocabulary codes (e.g., International Classification of Diseases (ICD) codes, Current Procedural Terminology (CPT) codes, etc.), and/or laboratory test results, free-text notes, etc. It may also include demographics information such as birth year, and sex. In one embodiment, EHR data 250 for patients in the cohort is directly retrieved from memory 224. In further embodiments, EHR data 250 is retrieved from an external data source by I/F 226, and then populated into memory 224 for use by controller 232. Individual health records within the health record data are associated with specific patients in the cohort via the use of uniquely associated identifiers within the health records and sequence data 240, which enables direct review of medical conditions for specific patients having specific variants.

Step 310 comprises controller 232 identifying a prevalence of the medical condition within the cohort. As a general process, this comprises determining a rate at which the medical condition is found within the cohort, such as by identifying medical vocabulary codes for the medical condition, for medical procedures that are performed in response to the medical condition, and/or for measurements that are used to delineate the medical condition from other medical conditions. In many instances, the specific medical condition being considered is already known a priori for the patient being analyzed (e.g., based on the gene that the variant is found within). Thus, the specific medical condition may be associated, a priori, with a specific set of codes across one or more medical vocabularies.

The prevalence of the medical condition within the cohort may be compared to the rate at which the medical condition is found within the general population, in order to form insights between the variant and the medical condition. Depending on the nature of the variant, it may be either positively or negatively associated with the medical condition.

In one embodiment, controller 232 identifies a first control set of patients that have variants in the gene with a well-established classification of pathogenic (a “positive control set”), and identifies a second control set of patients that have no non-synonymous variants in the gene (a “negative control set”). Health record data for patients in the first control set and second control set both are compared to health record data for patients in the cohort (those with the same variant being interpreted), to determine differences in prevalence of the medical condition between populations.

In step 312, controller 232 determines whether classification data 248 conflicts with a prevalence of the medical condition within the cohort. Classification data 248 conflicts with the prevalence in the cohort if it includes a classification for the variant as pathogenic or likely pathogenic, and the prevalence in the cohort is the same or less than in the general population. Similarly, classification data 248 conflicts with the prevalence in the cohort if it includes a classification for the variant as benign or likely benign, and the prevalence in the cohort is the greater than in the general population.

In a further embodiment, controller 232 calculates a likelihood (e.g., a p-value or odds ratio) that the classification data 248 for the variant is consistent with a prevalence of the medical condition within the cohort, and compares the likelihood to a threshold value such as classification threshold 252 stored in memory 224 (e.g., an odds ratio of two, an odds ratio of five, a p-value of 0.0001, a p-value of 0.95, etc.). In an event that the likelihood is less than the threshold value (e.g., if a p-value is below a threshold value or an odds ratio is above a threshold value), controller 232 determines that the classification data 248 for the variant conflicts with a prevalence of the medical condition within the cohort. Conversely, in an event that the likelihood is greater than the threshold value (e.g., as indicated by a p-value below a threshold value or an odds ratio above a threshold value), controller 232 determines that the classification data 248 for the variant does not conflict with a prevalence of the medical condition within the cohort.

In a further embodiment, controller 232 also determines whether data for the cohort indicates that the variant is pathogenic within an all-comers population. In this embodiment, controller 232 determines a p-value that there is a statistically different prevalence of the medical condition among patients with the variant than for the overall population. If the p-value is below a threshold, then the prevalence of the disease in those with the variants is statistically different from the overall population and that is evidence for pathogenicity. If the p-value is above the threshold, then there is little or no difference between those with the variant and the general population and it is evidence to indicate that the variant has no effect in relation to the disease.

In step 314, in an event that the classification data 248 for the variant conflicts with a prevalence of the medical condition within the cohort, controller 232 generates a recommendation to apply a classification to the variant that conforms with the prevalence of the medical condition within the cohort. For example, controller 232 may generate a recommendation to classify the variant as benign instead of VUS, when it appears that the medical condition is not more prevalent among carriers of the variant.

In step 316, in an event that the classification data 248 does not conflict with a prevalence of the medical condition within the cohort, controller 232 generates a recommendation to confirm a classification for the variant recited within the classification data 248. In this manner, controller 232 may help to confirm previous conclusions related to pathogenicity, further enhancing the certainty of a classification.

In step 318, controller 232 instructs I/F 226 to transmit guidelines 254 to provider client 210, to facilitate care or treatment of the patient based on the classification for the variant. Guidelines 254 may comprise standards of medical care for treatment related to the condition, and may vary depending on whether the variant is classified as PATH/LPATH, VUS, or BEN/LBEN. For example, in an event that the variant is classified as PATH/LPATH, guidelines 254 may recite standards of care for enhanced screening for the medical condition (e.g., a higher-than-normal incidence of imaging and/or blood testing), preventive care/treatments (e.g., blood pressure medication, a mastectomy, etc.), or lifestyle changes. In an event that the variant is classified as BEN/LBEN, guidelines 254 may indicate that practices used for the general population will be sufficient for the patient.

Method 300 provides a notable technical benefit by proactively identifying and compensating for selection bias that is likely to be present in classification data sets, especially those provided by clinical laboratories. By compensating for this error, especially when resolving VUS classifications into other classifications, health care providers receive actionable insights that facilitate patient care/treatment. Thus, personalized medicine for patients may be performed in a manner that is both more precise and more accurate, helping to ensure that patients receive the care that they need.

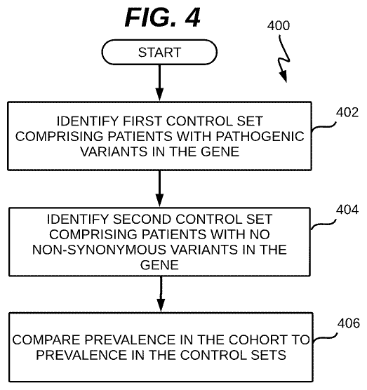

FIG. 4 is a flowchart depicting a method 400 for utilizing multiple control sets of patients to enhance classification of variants in an illustrative embodiment. Method 400 may be performed, for example, at steps 308, 310, and/or 312 of method 300 in order to provide insight into whether or not classification data 248 for a patient should be revised or enhanced.

Step 402 comprises controller 232 identifying a first control set of patients. The first control set of patients may include patients having variants classified as pathogenic in the same gene as the variant for the patient. However, the first control set need not be limited to patients having variants with well-established pathogenic interpretations. Patients from the first control set are selected from the same group that was used to source the patients of the cohort. For example, patients for both the first control set and the cohort may be selected from the same all-comers population genomics dataset, supplemented by EHR data for those patients to create a large scale clinicogenomic data set (e.g., representing hundreds of thousands of patients or more). In one embodiment, controller 232 applies further criteria for inclusion of patients in the first control set, requiring patients in the first control set to also have shared phenotypes or demographics (e.g., age or age range/bracket, sex, and/or ancestry) with the patient being considered. Filtering based on shared characteristics may help to improve the accuracy of results by having matched controls. For example, filtering may be used to ensure that not all controls (i.e., those with a P variant) are notably older (e.g., multiple decades older) than the patients in the cohort that have the variant of interest, as this would skew the control group towards a higher cancer rate, independent of genetics.

Step 404 comprises controller 232 identifying a second control set of patients that have no non-synonymous variants in the same gene as the variant for the patient. Patients from the second control set are selected from the same group that was used to source the patients of the cohort.

In further embodiments, one or more of the control sets are propensity matched against the cohort being studied. This helps to ensure that differences in prevalence of the disease between patients having the variant of interest and patients in the control sets are attributable to differences in genetics, rather than other factors. Specifically, propensity matching helps to reduce bias by accounting for confounding variables (e.g., age and sex) that may also have an impact on likelihood of the disease. By making control sets more closely resemble the cohort being studied, this helps to eliminate differences in confounding variables that could otherwise contribute to the disease being studied.

As used herein, propensity refers to a likelihood of having the disease, given specific values for covariates such as any suitable selection or combination of age, sex, weight, body mass index, pre-existing medical conditions, socioeconomic status, income, education, etc. Propensity matching attempts to compare groups having similar propensity scores in both the cohort being studied and the control sets, in order to eliminate differences in disease prevalence that would otherwise exist due to differences in covariates.

Depending on embodiment, propensity may be estimated using a scoring function, such as via logistic regression. Covariates that are expected or determined to be confounding may then be identified, and a propensity score may be calculated, such as via a p-value or odds ratio. In further embodiments, cohort members may be propensity-matched against individuals in one or more of the control sets, on an individualized one-to-one basis or on a group basis. This may be performed via techniques such as nearest neighbor matching, optimal full matching, caliper matching, radius matching, kernel matching, Mahalanobis metric matching, stratification matching, difference-in-differences matching, and/or exact matching.

After control sets have been populated, controller 232 may further confirm that values of covariates are similar between the cohort and the control sets, resulting in similar propensity scores between these groups.

With both control sets now defined, controller 232 proceeds in step 406 to compare prevalence of the medical condition within the cohort to prevalence in each of the control sets. The first control set indicates prevalence among those who have a pathogenic variant in the gene, while the second control set indicates prevalence among those who do not have a variant that is pathogenic or even possibly pathogenic in the gene. If the prevalence among the cohort is more statistically similar (e.g., average occurrence rate in the population, occurrence rate by a specific age, etc.) to the first control set than the second control set, this may be taken as evidence of pathogenicity. Alternatively, if the prevalence among the cohort is more statistically similar to the second control set than the first control set, this may be taken as evidence against pathogenicity. Controller 232 may determine a level of statistical similarity between populations by determining odds ratios or p-values for prevalence between the populations, and comparing those metrics.

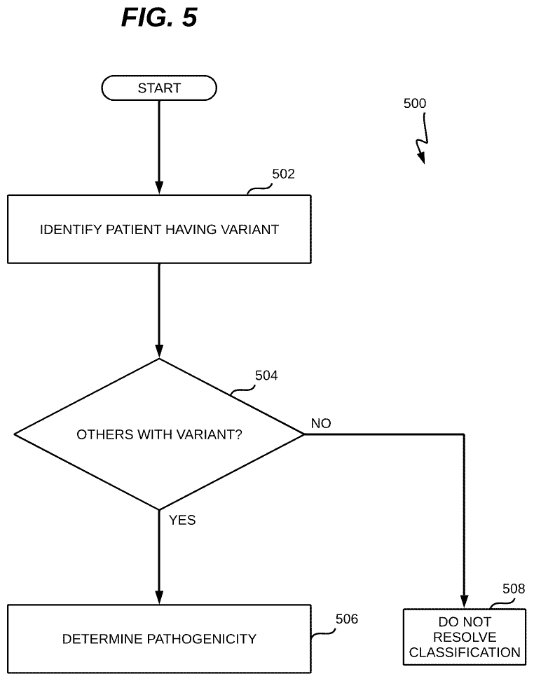

FIG. 5 is a flowchart depicting a method 500 for utilizing phenotype scores to enhance classification of variants in an illustrative embodiment. Method 500 may be performed, for example, at steps 308, 310, and/or 312 of method 300.

Step 502 includes identifying a patient. This may comprise selecting a patient awaiting variant classification, such as a patient that has been queued for variant classification.

Step 504 comprises determining whether other patients, within the same population that the cohort was selected from, have the same variant. Controller 232 may make this determination by reviewing genetic data for these persons. If there are other such patients, processing advances to step 506. Else, processing continues to step 508 where the classification is not resolved or otherwise changed.

Step 506 comprises determining pathogenicity for patients having the medical condition and the same variant, based on a calculated phenotype score. In one embodiment, this comprises calculating a phenotype score by fitting a logistic regression model according to the formula below, wherein B^{T}Xᵢ represents a weighted combination of phenotypes reported in health record data, and P(Zi=1) represents a likelihood of having a pathogenic or likely pathogenic variant in the gene under consideration:

logit(P(Zᵢ=1))=β^{T}Xᵢ (1)

Specific classifications for the variant may then be recommended based on the value of P(Zᵢ=1), where higher ranges of P(Zᵢ=1) are rated as more pathogenic. For example, in one embodiment, likelihoods cutoff of fifty percent, seventy-five percent, and ninety five percent may be used to define categories for rating variants as VUS, LPATH, and PATH, respectively.

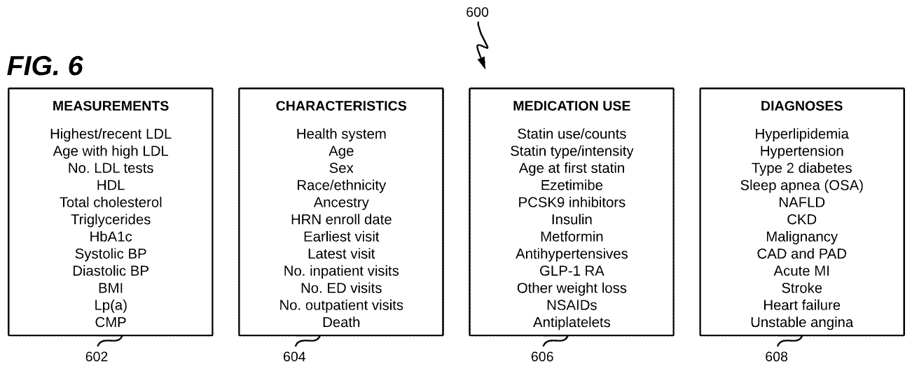

FIG. 6 is a block diagram 600 depicting categories of health record data in an illustrative embodiment. Specifically, diagram 600 variously depicts categories and corresponding types of data points relating to measurements 602 taken for a patient, characteristics 604 of a patient, medication use 606 by a patient, and diagnoses 608 for a patient (e.g., including diagnoses for specific medical conditions, such as diseases). These categories of data, and types of data points, may be processed by reference to medical codes, free-text notes, and/or other information within an EHR accessible to controller 232.

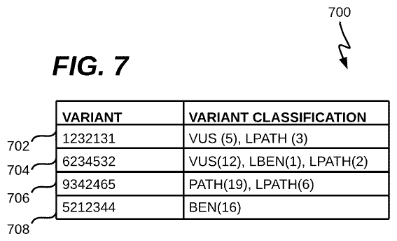

FIG. 7 is a block diagram depicting variant classification data 700 in an illustrative embodiment. In one embodiment, variant classification data 700 is received from a clinical laboratory that performs variant interpretation on a population of patients that are expected to have pathogenic variants. That is, patients referred to the clinical laboratory are referred because they are expected to be at risk of pathogenicity.

In this embodiment, variant classification data 700 includes a unique identifier for each of variants 702, 704, 706, and 708. Variant classification data 700 further recites categories/classifications that have been assigned to patients having each listed variant. This information may be utilized by controller 232 to determine whether or not a variant called for a patient would benefit from being resolved or enhanced via the techniques and methods described above.

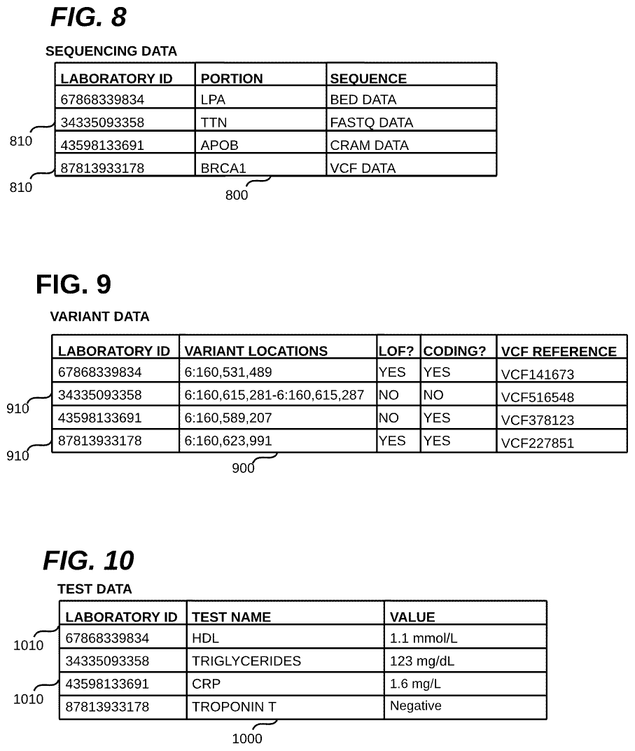

FIG. 8 is a table 800 that summarizes sequencing data for one or more genes for individuals in an illustrative embodiment. For example, table 800 may be one of many data structures stored in genomics server 220. In this embodiment, table 800 includes an entry 810 for each of multiple patients. Each entry 810 includes a unique identifier (e.g., LSID) for the corresponding patient, as well as an indication of the gene that the sequence data relates to. The portion of the genome that has been sequenced may comprise whole genome data, whole exome data, array data, data for a specific gene or portion of a gene, etc. Table 800 also indicates a format of the sequence data. Table 800 may be generated based on, or with reference to, sequences that have been alignment-enhanced via the processes discussed above.

FIG. 9 is a table 900 that summarizes variant data for individuals in an illustrative embodiment. In this embodiment, each entry 910 in table 900 reports a location (e.g., chromosomal coordinate) for each genetic variant, together with flags indicating whether the variant is a Loss of Function (LoF) variant or a coding variant. Table 900 further includes a VCF reference, which refers to the location and/or identifier of a VCF file that indicates the presence of the variant. The VCF file may be generated using data from the alignment enhancement processes discussed above. For example, alignment-enhanced data in a BAM, SAM, or CRAM file may include data used to generate the VCF file. Table 900 may be utilized by controller 232 of genomics server 220, in order to rapidly select and report diagnostic and care or treatment thresholds for a patient. Table 900 may be generated based on, or with reference to, sequences that have been alignment-enhanced via the processes discussed above.

FIG. 10 is a table 1000 that summarizes biomarker test data for individuals in an illustrative embodiment. Specifically, table 1000 summarizes test data pertaining to predetermined diseases for each of multiple patients in an illustrative embodiment. Each entry 1010 in table 1000 indicates an anonymized laboratory ID for a patient, a corresponding test name, and a corresponding value. Table 1000 may be created, for example, based on EHR data retrieved for patients. Laboratory IDs may be associated with EHR identifiers at genomics server 220 or provider client 210, in order to enable access to both health data and genomics data for a patient. Table 1000 may be used to enhance or provide context for genetic insights determined based on sequences that have been alignment-enhanced via the processes discussed above.

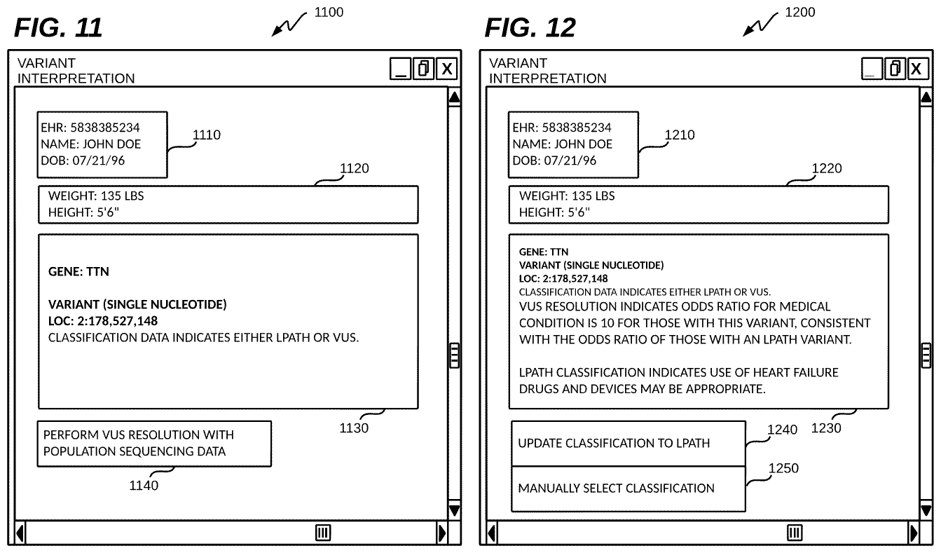

FIGS. 11-12 depict Graphical User Interfaces (GUIs) that facilitate the communication of information related to variant classifications in illustrative embodiments. Specifically, FIG. 11 depicts a GUI 1100 which reports a variant, as well as a determination that the variant is either classified as a VUS or is subject to multiple classifications. GUI 1100 includes element 1110 for identifying information for the patient, element 1120 for phenotype information for the patient, and element 1130 for reporting variant information for the patient. Element 1140 comprises an interactive element (e.g., a button) which in this embodiment triggers the variant classification enhancement processes discussed in method 300.

FIG. 12 depicts a GUI 1200 which reports enhanced classification for a variant, as well as care/treatment options or recommendations in light of the enhanced classification. GUI 1200 includes element 1210 for reporting identifying information for the patient, element 1220 for phenotype information for the patient, and element 1230 for reporting variant information for the patient as well as care/treatment information. Element 1240 comprises an interactive element (e.g., a button) which in this embodiment applies the enhanced variant classification to the patient. Element 1250 comprises an interactive element that permits manually selecting the variant classification for the patient.

Any of the various computing and/or control elements shown in the figures or described herein may be implemented as hardware, as a processor implementing software or firmware, or some combination of these. For example, an element may be implemented as dedicated hardware. Dedicated hardware elements may be referred to as “processors,” “controllers,” or some similar terminology. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, a network processor, application specific integrated circuit (ASIC) or other circuitry, field programmable gate array (FPGA), read only memory (ROM) for storing software, random access memory (RAM), non-volatile storage, logic, or some other physical hardware component or module.

In one embodiment, instructions stored on a computer readable medium direct a computing system of any of the devices and/or servers discussed herein, such as genomics server 220, to perform the various operations disclosed herein. In some embodiments, all or portions of these operations may be implemented in a networked computing environment, such as a cloud computing system. Cloud computing often includes on-demand availability of computer system resources, such as data storage (cloud storage) and computing power, without direct active management by an entity. Cloud computing relies on the sharing of resources, and generally includes on-demand self-service, broad network access, resource pooling, rapid elasticity, and measured service.

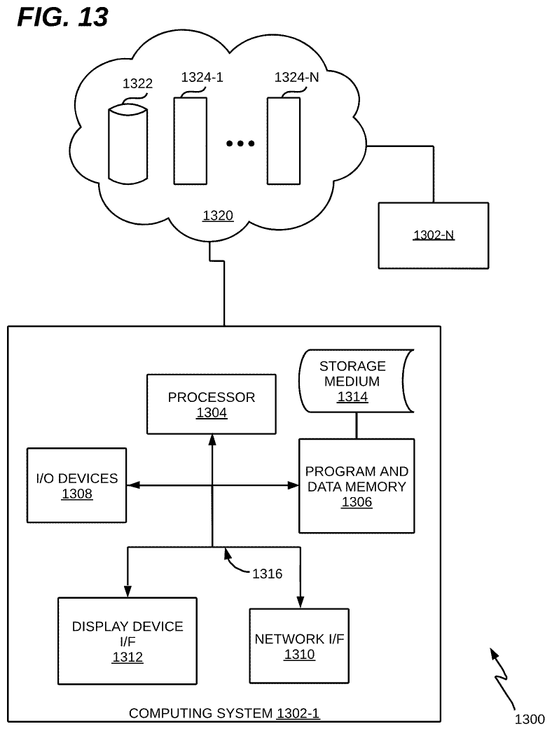

FIG. 13 depicts one illustrative cloud computing system 1300 operable to perform the above operations by executing programmed instructions tangibly embodied on one or more computer readable storage mediums. The cloud computing system 1300 generally includes the use of a network of remote servers hosted on the internet to store, manage, and process data, rather than a local server or a personal computer (e.g., in the computing systems 1302-1-1302-N). Cloud computing enables users to use infrastructure and applications via the internet, without installing and maintaining them on-premises. In this regard, the cloud computing network 1320 may include virtualized information technology (IT) infrastructure (e.g., servers 1324-1-1324-N, the data storage module 1322, operating system software, networking, and other infrastructure) that is abstracted so that the infrastructure can be pooled and/or divided irrespective of physical hardware boundaries. In some embodiments, the cloud computing network 1320 can provide users with services in the form of building blocks that can be used to create and deploy various types of applications in the cloud on a metered basis.

Various components of the cloud computing system 1300 may be operable to implement the above operations in their entirety or contribute to the operations in part. For example, a computing system 1302-1 may be used to perform analysis of gene sequencing data, and then store that analysis along with the gene sequencing data in a data storage module 1322 (e.g., a database) of a cloud computing network 1320. Various computer servers 1324-1-1324-N of the cloud computing network 1320 may be used to operate on the gene sequencing data and/or transfer the gene sequencing analysis and/or the gene sequencing data to another computing system 1302-N.

Some embodiments disclosed herein may utilize instructions (e.g., code/software) accessible via a computer-readable storage medium for use by various components in the cloud computing system 1300 to implement all or parts of the various operations disclosed hereinabove. Examples of such components include the computing systems 1302-1-1302-N.

Exemplary components of the computing systems 1302-1-1302-N may include at least one processor 1304, a computer readable storage medium 1314, program and data memory 1306, input/output (I/O) devices 1308, a display device interface 1312, and a network interface 1310. For the purposes of this description, the computer readable storage medium 1314 comprises any physical media that is capable of storing a program for use by the computing system 1302. For example, the computer-readable storage medium 1314 may be an electronic, magnetic, optical, electromagnetic, infrared, semiconductor device, or other non-transitory medium. Examples of the computer-readable storage medium 1314 include a solid-state memory, a magnetic tape, a removable computer diskette, a random access memory (RAM), a read-only memory (ROM), a rigid magnetic disk, and an optical disk. Some examples of optical disks include Compact Disk-Read Only Memory (CD-ROM), Compact Disk Read/Write (CD-R/W), Digital Versatile Disc (DVD), and Blu-Ray Disc.

The processor 1304 is coupled to the program and data memory 1306 through a system bus 1316. The program and data memory 1306 include local memory employed during actual execution of the program code, bulk storage, and/or cache memories that provide temporary storage of at least some program code and/or data in order to reduce the number of times the code and/or data are retrieved from bulk storage (e.g., a hard disk drive, a solid state drive, or the like) during execution.

Input/output or I/O devices 1308 (including but not limited to keyboards, displays, touchscreens, microphones, pointing devices, etc.) may be coupled either directly or through intervening I/O controllers. Network adapter interfaces 1310 may also be integrated with the system to enable the computing system 1302 to become coupled to other computing systems or storage devices through intervening private or public networks. The network adapter interfaces 1310 may be implemented as modems, cable modems, Small Computer System Interface (SCSI) devices, Fibre Channel devices, Ethernet cards, wireless adapters, etc. Display device interface 1312 may be integrated with the system to interface to one or more display devices, such as screens for presentation of data generated by the processor 1304.

What is claimed is:

1. A system for enhancing classification of variants in a field of genetics, the system comprising:
an interface configured to retrieve genetic information that corresponds with a patient and that calls a variant within a gene related to a medical condition, and to retrieve classification data for the variant; and
a controller configured to identify a cohort of patients that have the variant and are not included within the classification data, to retrieve health record data related to the medical condition for the patients in the cohort, and to identify a prevalence of the medical condition within the cohort;
the controller further configured, in an event that the classification data conflicts with a prevalence of the medical condition within the cohort, to generate a recommendation to apply a classification to the variant that conforms with the prevalence of the medical condition within the cohort;
the controller further configured, in an event that the classification data does not conflict with a prevalence of the medical condition within the cohort, to generate a recommendation to confirm a classification for the variant recited within the classification data;
the controller further configured to provide a recommendation to treat the patient based on the classification for the variant.

2. The system of claim 1, wherein:
the controller is further configured to identify a first control set of patients having variants in the gene that are classified as pathogenic, and to identify a second control set of patients that have no non-synonymous variants in the gene.

3. The system of claim 1 wherein:
the controller is further configured to calculate a likelihood that the classification data is consistent with a prevalence of the medical condition within the cohort, and to compare the likelihood to a threshold value;
in an event that the likelihood is less than the threshold value, the controller is further configured to determine that the classification data conflicts with a prevalence of the medical condition within the cohort; and
in an event that the likelihood is greater than the threshold value, the controller is further configured to determine that the classification data does not conflict with a prevalence of the medical condition within the cohort.

4. The system of claim 1 wherein:
the classification data for the variant reports the variant as a Variant of Uncertain Significance (VUS).

5. The system of claim 1 wherein:
the classification data for the variant reports the variant as having multiple classifications.

6. The system of claim 1 wherein:
the genetic information is reported in a Variant Call Format (VCF) file for the patient.

7. The system of claim 1 wherein:
the health record data comprises Electronic Health Record (EHR) data for the patients.

8. A method for enhancing classification of variants in a field of genetics, the method comprising:
retrieving genetic information that corresponds with a patient and that calls a variant within a gene related to a medical condition;
retrieving classification data for the variant;
identifying a cohort of patients that have the variant and are not included within the classification data;
retrieving health record data related to the medical condition for the patients in the cohort;
identifying a prevalence of the medical condition within the cohort;
in an event that the classification data conflicts with a prevalence of the medical condition within the cohort:
generating a recommendation to apply a classification to the variant that conforms with the prevalence of the medical condition within the cohort;
in an event that the classification data does not conflict with a prevalence of the medical condition within the cohort:
generating a recommendation to confirm a classification for the variant recited within the classification data; and
providing a recommendation to treat the patient based on the classification for the variant.

9. The method of claim 8, further comprising:
identifying a first control set of patients having variants in the gene that are classified as pathogenic; and
identifying a second control set of patients that have no non-synonymous variants in the gene.

10. The method of claim 8 further comprising:
calculating a likelihood that the classification data is consistent with a prevalence of the medical condition within the cohort;
comparing the likelihood to a threshold value;
in an event that the likelihood is less than the threshold value, determining that the classification data conflicts with a prevalence of the medical condition within the cohort; and
in an event that the likelihood is greater than the threshold value, determining that the classification data does not conflict with a prevalence of the medical condition within the cohort.

11. The method of claim 8 wherein:
the classification data for the variant reports the variant as a Variant of Uncertain Significance (VUS).

12. The method of claim 8 wherein:
the classification data for the variant reports the variant as having multiple classifications.

13. The method of claim 8 wherein:
the genetic information is reported in a Variant Call Format (VCF) file for the patient.

14. The method of claim 8 wherein:
the health record data comprises Electronic Health Record (EHR) data for the patients.

15. A non-transitory computer readable medium embodying programmed instructions which, when executed by a processor, are operable for performing a method for enhancing classification of variants in a field of genetics, the method comprising:
retrieving genetic information that corresponds with a patient and that calls a variant within a gene related to a medical condition;
retrieving classification data for the variant;
identifying a cohort of patients that have the variant and are not included within the classification data;
retrieving health record data related to the medical condition for the patients in the cohort;
identifying a prevalence of the medical condition within the cohort;
in an event that the classification data conflicts with a prevalence of the medical condition within the cohort:
generating a recommendation to apply a classification to the variant that conforms with the prevalence of the medical condition within the cohort;
in an event that the classification data does not conflict with a prevalence of the medical condition within the cohort:
generating a recommendation to confirm a classification for the variant recited within the classification data; and
providing a recommendation to treat the patient based on the classification for the variant.

16. The non-transitory computer readable medium of claim 15, wherein the method further comprises:
identifying a first control set of patients having variants in the gene that are classified as pathogenic; and
identifying a second control set of patients that have no non-synonymous variants in the gene.

17. The non-transitory computer readable medium of claim 15, wherein the method further comprises:
calculating a likelihood that the classification data is consistent with a prevalence of the medical condition within the cohort;
comparing the likelihood to a threshold value;
in an event that the likelihood is less than the threshold value, determining that the classification data conflicts with a prevalence of the medical condition within the cohort; and
in an event that the likelihood is greater than the threshold value, determining that the classification data does not conflict with a prevalence of the medical condition within the cohort.

18. The non-transitory computer readable medium of claim 15, wherein:
the classification data for the variant reports the variant as a Variant of Uncertain Significance (VUS).

19. The non-transitory computer readable medium of claim 15, wherein:
the classification data for the variant reports the variant as having multiple classifications.

20. The non-transitory computer readable medium of claim 15, wherein:
the genetic information is reported in a Variant Call Format (VCF) file for the patient.
